(19) United States
(12) Patent Application Publication
(10) Pub. No.: US 2002/0015695 A1
(43) Pub. Date: Feb. 7, 2002

(54) Herpesviral vectors for gene delivery

(76) Inventors: Stacey Efstathiou, Cambridge (GB); Robin Henry Lachmann, Cambridge (GB); Cinzia Giuseppina Scarpini, Cambridge (GB)

Correspondence Address: KLARQUIST SPARKMAN CAMPBELL LEIGH & WHINSTON, LLP, One World Trade Center, Suite 1600, 121 S.W. Salmon Street, Portland, OR, 97204, US

(21) Appl. No.: 09/778,390

(22) Filed: Feb. 6, 2001

Related U.S. Application Data
(63) Continuation of application No. 09/365,654, filed on Aug. 2, 1999, now abandoned.

(30) Foreign Application Priority Data
Jul. 31, 1998 (GB) 9816775.2

(51) Int. Cl. A61K 48/00; C12N 15/861

(52) U.S. Cl. 424/93.21; 435/456; 435/235.1

(57) ABSTRACT

A mutant herpesvirus in which (a) an early gene encoding a function required for expression of late gene products has been inactivated, e.g. by deletion; and in which (b) a viral gene essential for production of infectious new virus particles has been inactivated, preferably by deletion; and in which (c) a gene to be delivered to a target cell has been inserted together with regulatory elements for its expression in a target cell, can be used as a gene delivery vector for gene delivery e.g. to central nervous system cells.

### FIELD OF THE INVENTION

This invention relates to herpesviral vectors and their production and use, e.g. for gene therapy and related purposes of gene delivery.

### BACKGROUND TO THE INVENTION

EP 0 453 242 (Gen. Hosp. Corp: Breakefield & Martuza) describes herpesvirus vectors based on HSV1 with a degree of replication-defectiveness, based on mutations of the viral TK and certain other genes, intended for CNS gene therapy, e.g. with a gene encoding HPRT enzyme.

A replication-defective virus according to U.S. Pat. No. 5,658,724 (Univ Pittsburgh: NA DeLuca) carries plural deletions of immediate early genes encoding ICP4 and ICP27 and is intended for gene therapy. VP16-mutant replication-defective viruses such as in1814 (see Harris, R A and C M Preston (1991) encode mutant forms of VP16 protein. WO 96/04395 (Lynxvale Ltd: P Speck) describes plural-mutant virus of reduced lytic effect, especially for use as a gene vector.

It has been reported that certain highly attenuated deletant herpesviruses are safe for intracerebral administration: e.g. a gH- TK- tsK triple mutant (MG Kaplitt et al, 1994 PNAS 91:8979-8983). However, such a mutant does give rise to expression of IE gene products and it has also been considered that IE gene expression alone can produce cytotoxicity (D R Jamieson et al, 1995 J gen Virol 76:1417-1431; N Wu et al, 1996 J Virol 70:6358-6369).

Accordingly, it remains desirable in the inventors' view to provide further mutant viruses that can effectively deliver genes as vectors, e.g. to CNS or non-neuronal tissue, but are of satisfactorily low toxicity.

### SUMMARY AND DESCRIPTION OF THE INVENTION

This invention provides use of a gene delivery vector based on a mutant herpesvirus in which (a) an early gene encoding a function that can be required for expression of late gene products (e.g. a gene that takes part in viral DNA replication in non-dividing cells such as CNS cells, e.g. neurones, and participates in late-gene expression in such cells, encoding for example TK, or encoding RNP subunit 1 or 2) has been inactivated, e.g. by deletion; and in which (b) a gene essential for production of infectious new virus particles has been inactivated, preferably by deletion; and in which (c) a gene to be delivered to a target cell has been inserted together with regulatory elements for its expression in a target cell, e.g. inserted at the site of deletion of the essential gene, e.g. a gene encoding an essential glycoprotein for example gH.

In certain useful embodiments of the invention, the vector does not lack the function of any of the normal herpesviral immediate early genes.

A useful example of a gene delivery vector according to the invention is a herpes simplex virus (type 1 or 2) which is TK negative, gH negative, and carries a therapeutic gene such as HPRT or any of a number of other examples of ‘cargo’ genes as detailed below.

Specification WO 96/26267 (Cantab Pharmaceuticals: MEG Boursnell et al) discloses at page 28 a virus constructed as an intermediate in the preparation of further virus vectors, which lacks both gH and TK gene functionality, in addition a lacz cassette at the locus of the deleted gH gene. Such a virus can be used in an example of the present invention as the basis of a virus vector for delivery of ‘cargo’ genes, e.g. to the central nervous system, the ‘cargo’ gene being placed in the position of the lacz cassette by per-se known manipulations.

Such a vector can be used for gene delivery to a CNS target with usefully little neurotoxicity. The low level of neurotoxicity experienced with this example is surprising, given that TK- HSV mutants, while known to be of reduced neurovirulence compared with wild-type virus, still have been considered too toxic for use as gene delivery vectors.

A further example of a virus vector that can be used according to the invention to give usefully low CNS toxicity can be made by recombination of any suitable herpes simplex virus, e.g. virus L beta A (designating a virus vector described in WO 97/20935, CU Tech Services Ltd: S Efstathiou & RH Lachmann, incorporated herein by reference), containing all potential downstream and upstream long-term LAP regulatory regions linked to an IRES-lacZ cassette; using for said recombination for example a plasmid pIMMB34 as described in WO 96/26267 (Cantab Pharmaceuticals: MEG Boursnell et al) to delete the TK and gH genes.

A vector to be used according to the invention can carry any of a variety of desired genes for delivery to a target cell or tissue, e.g. genes/gene products, and methods of delivery, as mentioned in WO 97/20935 (CU Tech Services: Efstathiou et al) or in WO 96/27672 (Fink & Glorioso), both of which specifications are hereby incorporated by reference in their entirety. A vector for use according to the invention can encode gene products of any of the kinds mentioned in WO 96/26267 (Cantab Pharmaceuticals: MEG Boursnell et al), or in WO 96/27672 cited above, that are to be delivered to target cells; and as a base virus mutant for the construction of mutant virus vectors carrying synthetic (e.g. semisynthetic) latency-active regulatory sequences. Heterologous genes that can be included as ‘cargo’ genes in virus vectors according to examples of the invention can encode for example products selected from neurotrophic factors, such as GDNF, CTNF, and BDNF, and nerve growth factors such as NGF. Further examples of useful ‘cargo’ genes are those encoding hexosaminidase (known in connection with Tay-Sachs and Sandhoff diseases), arylsulphatase A (known in connection with metachromatic leucodystrophy), the NPC1 gene (known in connection with Niemann Pick Disease type C) and glucocerebrosidase (known in connection with Gaucher's disease).

Such a vector can for example be made and used on the basis of modification of the per-se known procedures e.g. as used for making mutant viruses in the references cited herein.

Thus it can be seen that the invention provides inter alia herpesviral vectors for gene delivery with reduced toxicity but without need for immediate-early gene inactivations.

The invention is susceptible to a variety of modifications and variations as will be apparent to those skilled in the art, and the present disclosure extends to combinations and subcombinations of the features mentioned or described herein and in the cited documents which are hereby incorporated by reference in their entirety for all purposes.

1. Use of a mutant herpesvirus in which (a) TK or other early gene encoding a function that can be required for expression of late gene products has been inactivated, e.g. by deletion; and in which (b) a viral gene essential for production of infectious new virus particles has been inactivated, preferably by deletion; and in which (c) a gene to be delivered to a target cell has been inserted together with regulatory elements for its expression in a target cell, as a gene delivery vector, e.g. for gene delivery to central nervous system cells.

2. Use according to claim 1, wherein gene (a) encodes TK, or RNP subunit 1 or 2.

3. Use according to claim 1, wherein gene (c) is inserted at the site of deletion of the essential viral gene, e.g. a gene encoding an essential glycoprotein, for example gH.
